(19) United States
(12) Patent Application Publication
(10) Pub. No.: US 2025/0152166 A1
(43) Pub. Date: May 15, 2025

(54) KNOTLESS SOFT TISSUE DEVICES AND TECHNIQUES

(71) Applicant: Biomet Sports Medicine, LLC, Warsaw, IN (US)

(72) Inventors: Daniel Norton, Warsaw, IN (US); Kevin T. Stone, Winona Lake, IN (US)

(21) Appl. No.: 19/023,072

(22) Filed: Jan. 15, 2025

Related U.S. Application Data
(63) Continuation of application No. 18/107,313, filed on Feb. 8, 2023, now Pat. No. 12,251,100.
Application No. 18/107,313 is a continuation of application No. 16/927,382, filed on Jul. 13, 2020, now Pat. No. 11,648,004.
Application No. 16/927,382 is a continuation of application No. 16/160,501, filed on Oct. 15, 2018, now Pat. No. 10,806,443.
Application No. 16/160,501 is a continuation of application No. 14/137,350, filed on Dec. 20, 2013, now Pat. No. 10,136,886.

(51) Int. Cl. A61B 17/06; A61B 17/04; A61B 90/92; A61B 90/94

(52) U.S. Cl. CPC A61B 17/06166; A61B 17/0401; A61B 17/0485; A61B 17/0487; A61B 2017/0404; A61B 2017/0406; A61B 2017/0417; A61B 2017/0458; A61B 2017/0495; A61B 90/92; A61B 90/94

(57) ABSTRACT

A tissue fixation device including a self-locking construct having a first loop, a second loop, and a first free end. A first tail is coupled to, and extends from, the first loop. A second tail is coupled to, and extends from, the second loop. An anchor is coupled to one of the self-locking construct or the first tail such that one of the self-locking construct or the first tail extend through the anchor.

### FIELD

The present disclosure relates to knotless soft tissue devices and techniques.

### BACKGROUND

This section provides background information related to the present disclosure, which is not necessarily prior art.

Arthroscopic procedures often include sutures and anchors to secure soft tissue to bone, secure bone pieces together, and to secure separated portions of soft tissue together. Despite their widespread use, sutures and suture anchors, as well as methods for their use, can be improved. For example, tying sutures into knots may be very time consuming and difficult to perform, particularly inside the joint space. As a result, the cost of the procedure may increase and the capacity of the surgeon may be limited. Furthermore, the strength of the repair may be limited by the strength of the knot. The methods and devices disclosed herein address these issues and numerous others.

### SUMMARY

This section provides a general summary of the disclosure, and is not a comprehensive disclosure of its full scope or all of its features.

The present teachings provide for a tissue fixation device including a self-locking construct having a first loop, a second loop, and a first free end. A first tail is coupled to, and extends from, the first loop. A second tail is coupled to, and extends from, the second loop. An anchor is coupled to one of the self-locking construct or the first tail such that one of the self-locking construct or the first tail extend through the anchor.

The present teachings also provide for a tissue fixation device including a self-locking construct having a first loop and a free end. A first tail is coupled to, and extends from, the first loop. A second tail is coupled to, and extending from, the self-locking construct. An anchor is coupled to the first tail such that the self-locking construct extends through the anchor.

The present teachings also provide for a method for securing soft tissue to bone. The method includes implanting a first anchor into bone, the first anchor coupled to one of a first loop of a first self-locking construct or a first tail mounted to the first loop. The first tail and a portion of the first loop are passed through the tissue. The first tail is passed through a second loop and the first loop is partially passed through the second loop on a side of the tissue opposite to the bone. A first free end of the first self-locking construct is pulled to close the first loop.

Further areas of applicability will become apparent from the description provided herein. The description and specific examples in this summary are intended for purposes of illustration only and are not intended to limit the scope of the present disclosure.

The drawings described herein are for illustrative purposes only of selected embodiments and not all possible implementations, and are not intended to limit the scope of the present disclosure.

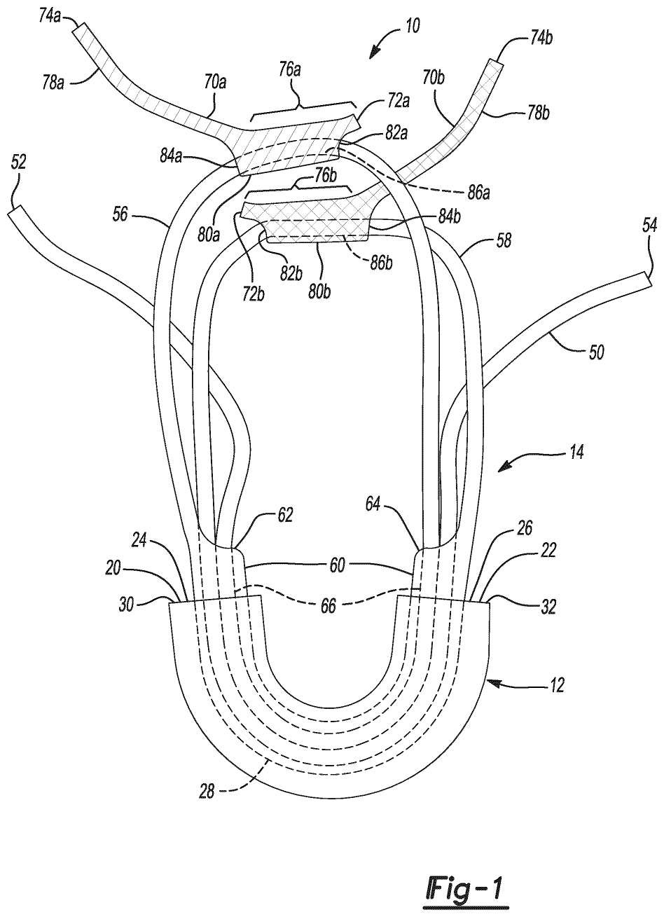

FIG. 1 illustrates a tissue fixation device according to the present teachings;

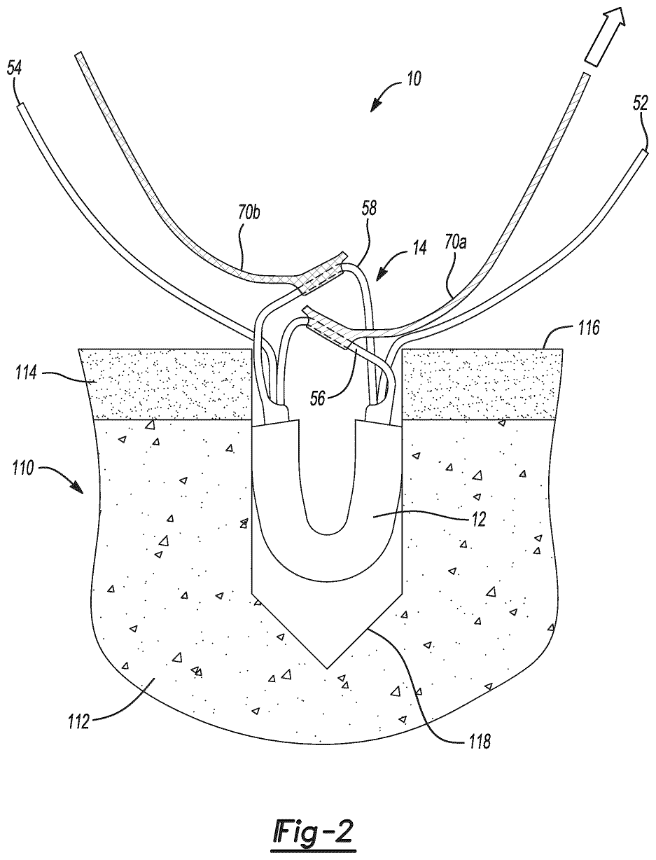

FIG. 2 illustrates the tissue fixation device of FIG. 1 in bone;

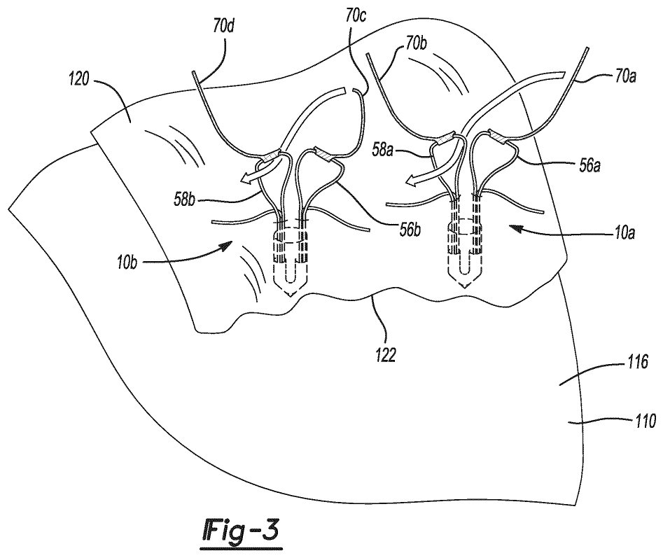

FIG. 3 illustrates two of the tissue fixation devices of FIG. 1 implanted in bone to affix tissue relative to the bone;

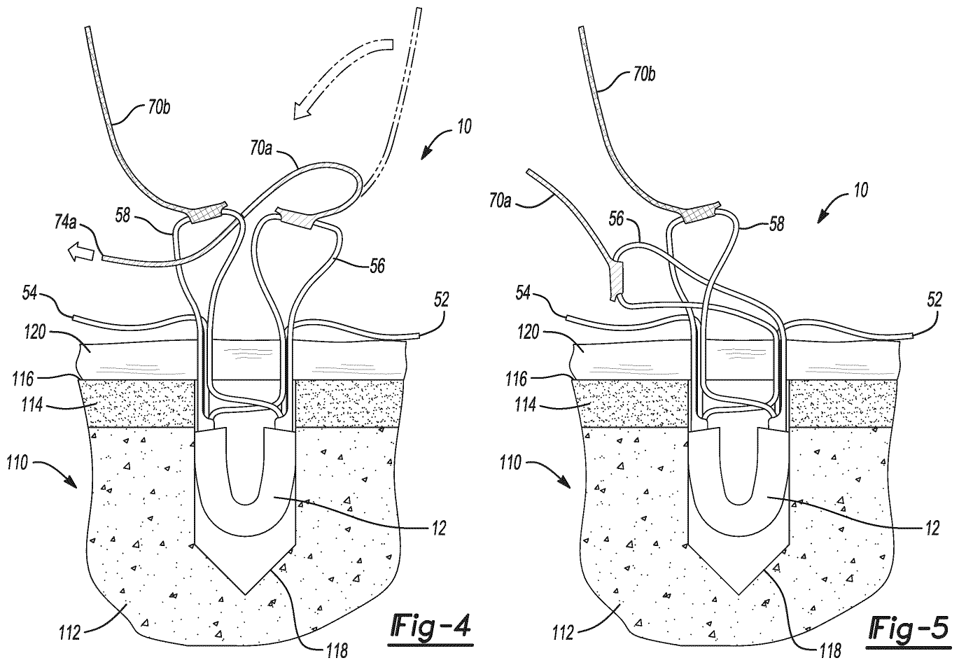

FIGS. 4-8 illustrate use of the tissue fixation device of FIG. 1 to fix the tissue to bone;

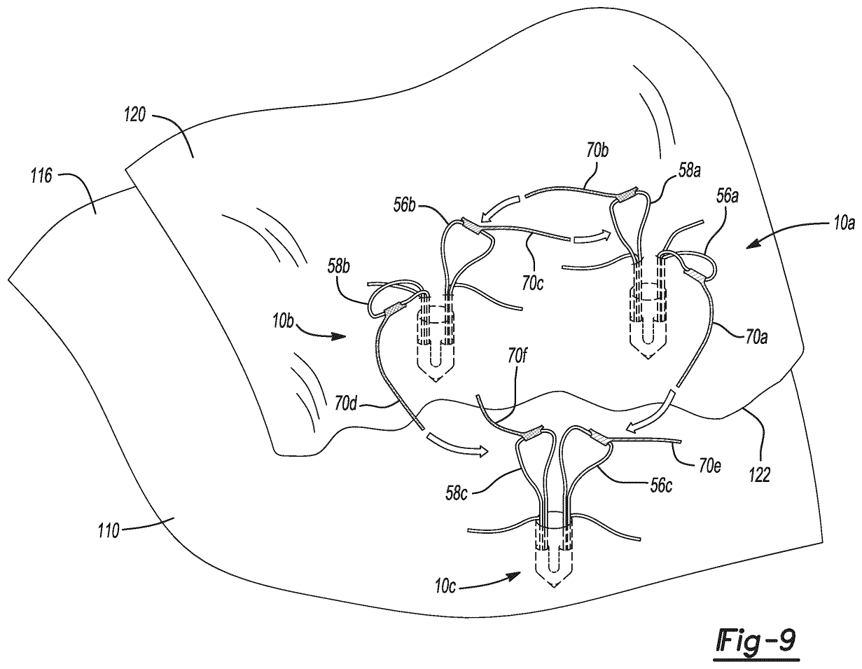

FIG. 9 illustrates three of the tissue fixation devices of FIG. 1 implanted in bone for affixing tissue to bone;

FIG. 10 illustrates the tissue fixation device of FIG. 1 implanted in bone, and an additional flexible anchor being added to the fixation device;

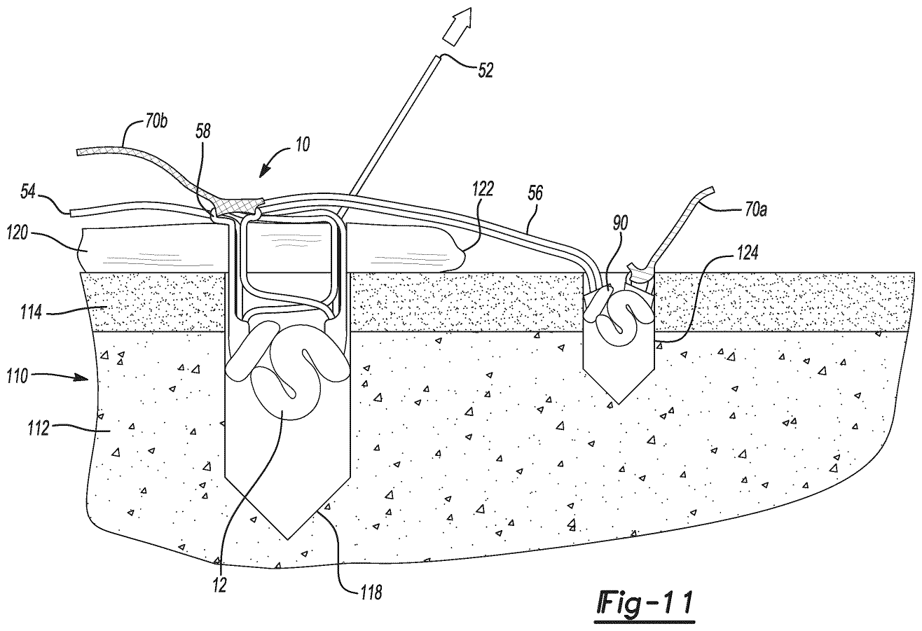

FIG. 11 illustrates the tissue fixation device of FIG. 1 implanted in bone to secure tissue to bone, and the additional anchor of FIG. 10 implanted in bone to facilitate tissue fixation and tensioning;

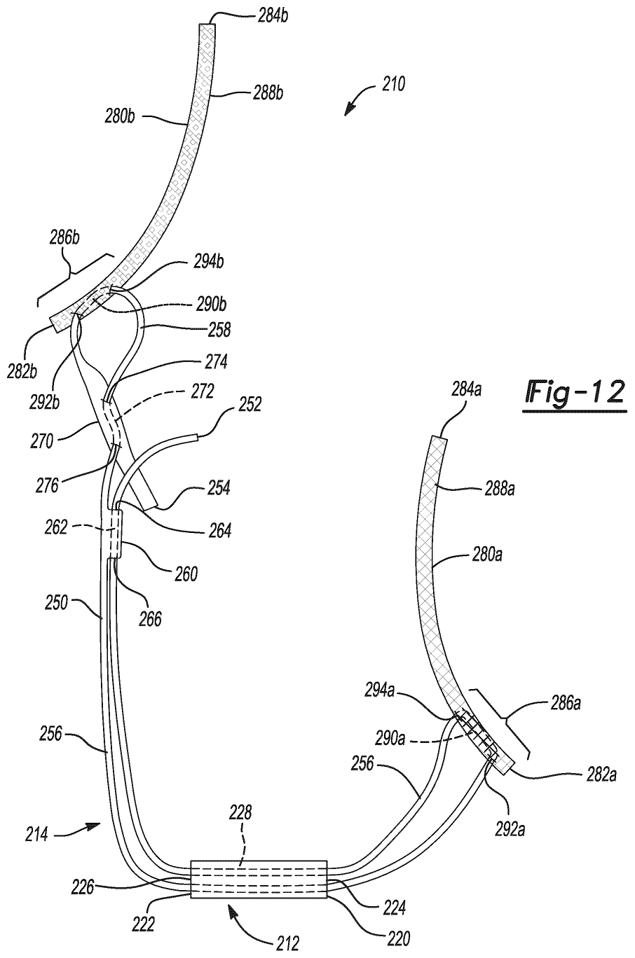

FIG. 12 illustrates another tissue fixation device according to the present teachings, with an anchor positioned on a suture construct of the tissue fixation device;

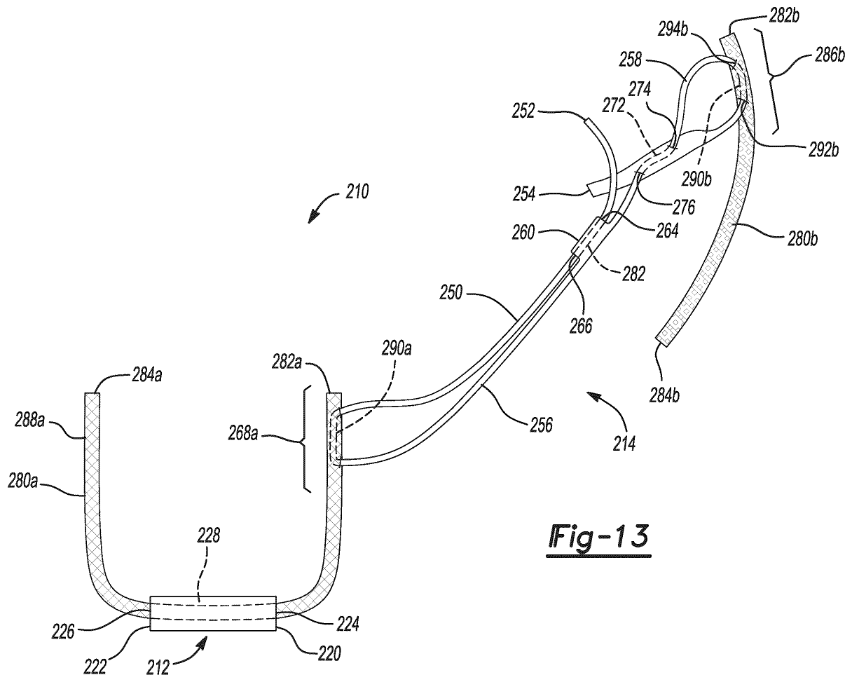

FIG. 13 illustrates the tissue fixation device of FIG. 12 with the anchor positioned on a tail of the tissue fixation device;

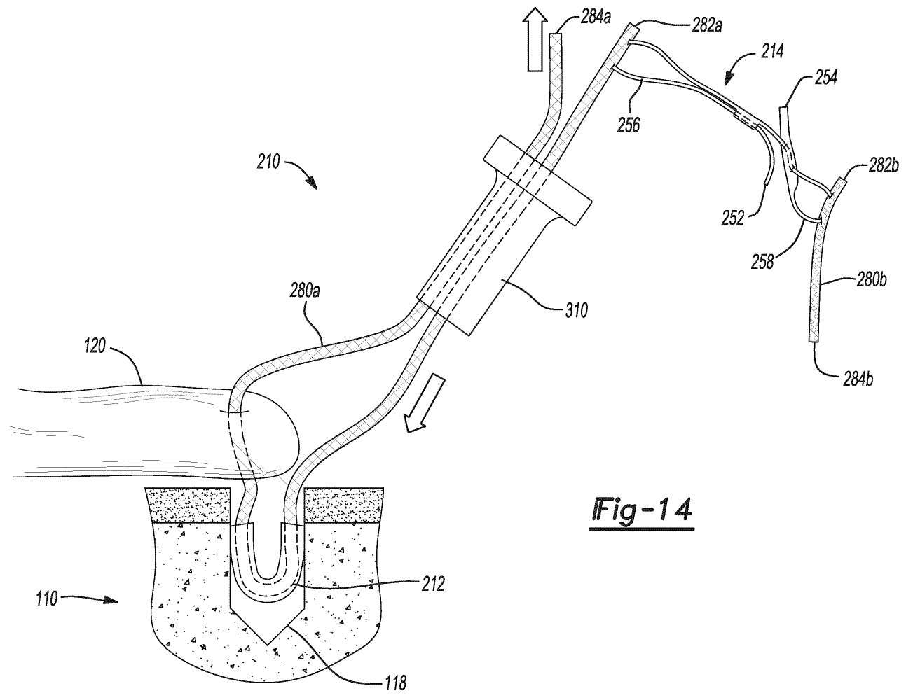

FIGS. 14-18 illustrate use of the tissue fixation device of FIG. 13 to affix tissue to bone;

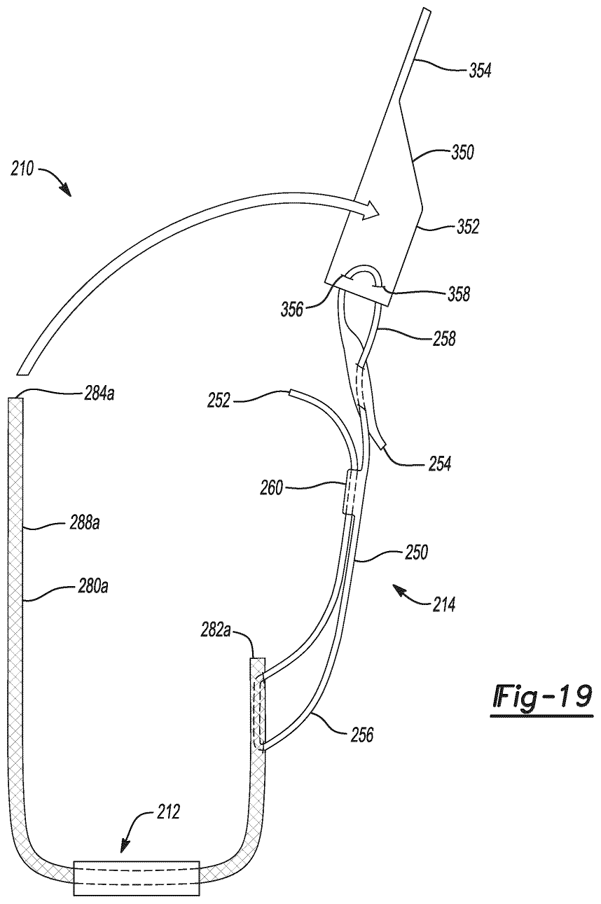

FIG. 19 illustrates the tissue fixation device of FIG. 13 including a braided strip;

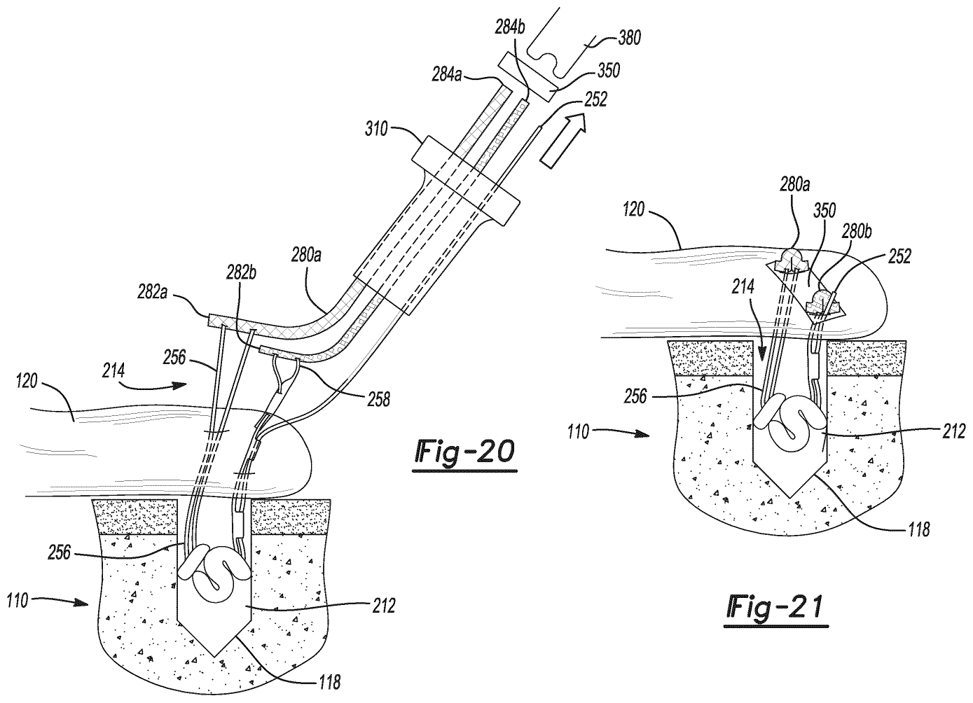

FIGS. 20 and 21 illustrate use of a braided strip to facilitate securing tissue to bone; and

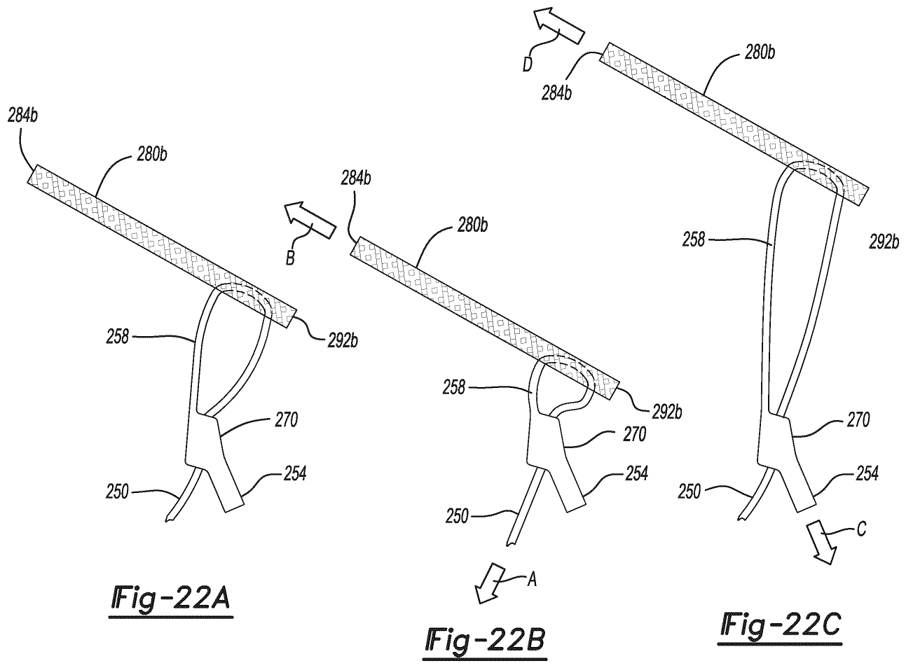

FIGS. 22A-22C illustrate a tail of either one of the tissue fixation devices of FIGS. 12 and 13 coupled to a loop of the tissue fixation devices, and use of the tail to open the loop.

Corresponding reference numerals indicate corresponding parts throughout the several views of the drawings.

### DETAILED DESCRIPTION

Example embodiments will now be described more fully with reference to the accompanying drawings.

With initial reference to FIG. 1, a tissue fixation device according to the present teachings is generally illustrated at reference numeral 10. Although the tissue fixation device 10 is generally described herein as configured to secure tissue to bone, the tissue fixation device 10 can also be used to, for example, secure two bones or bone portions together, as well as two tissues or tissue portions together. The tissue fixation device 10 generally includes a soft or flexible anchor 12 and a self-locking construct 14.

The anchor 12 includes a first anchor end 20 and a second anchor end 22, which is opposite to the first anchor end 20. The anchor 12 can be an elongate member having a sleeve or tubular configuration. The anchor 12 thus defines a first opening 24 at the first anchor end 20, and defines a second opening 26 at the second anchor end 22. The anchor 12 further defines an anchor internal passage 28 extending between the first opening 24 and the second opening 26. At the first anchor end 20 is a first end surface 30, and at the second anchor end 22 is a second end surface 32. As described herein, the first and the second end surfaces 30 and 32 are configured to abut an interior surface of cortical bone in order to facilitate retention of the tissue fixation device 10 within a bone hole.

In place of the anchor 10, any suitable soft or hard anchor can be used, such as any of the soft or hard anchors described in U.S. application Ser. No. 13/098,927 (“'927 Application”) titled “Method and Apparatus for Soft Tissue Fixation,” filed on May 2, 2011, and assigned to Biomet Sports Medicine, LLC, which is incorporated herein by reference. In place of the anchor 10 can also be any of the anchors described in U.S. Pat. No. 8,562,647 ('647 Patent “) titled “Method and Apparatus for Securing Soft Tissue to Bone,” issued on Oct. 22, 2013, and assigned to Biomet Sports Medicine, LLC, which is incorporated herein by reference. The anchor 12 can be made of any suitable material, such as resorbable or non-resorbable materials, including braided suture, sponges, and sponge-like materials, including braided suture, sponges, and sponge-like materials in solid form, perforated materials, woven/braided from biocompatible materials or fibers, such as, for example, polymer, polyester, polyethylene, cotton, silk, or other natural or synthetic materials.

The self-locking construct 14 can be made of any suitable material, such as a flexible material including suture, such as a suture strand 50. When the construct 14 is made of suture, any suitable suture can be used. For example, a braided hollow-core suture strand 50 can be used. Any suitable braided suture can be used, such as any of the braided sutures disclosed in U.S. patent application Ser. No. 12/915,962 titled “Method and Apparatus for Securing Soft Tissue to Bone,” which was filed on Oct. 29, 2010, published as Publication No. 2011/0098727 on Apr. 28, 2011, and is assigned to Biomet Sports Medicine, LLC. Although the construct 14 can be made of any suitable material in addition to suture, the construct 14 will generally be described herein as a construct.

The construct 14 generally includes a first end 52 and a second end 54 of the suture strand 50. The suture strand 50 is arranged to provide a self-locking, first adjustable loop 56 and a self-locking, second adjustable loop 58. The suture strand 50 includes a portion 60 between the first end 52 and the second end 54. The portion 60 itself includes a first end 62 and a second end 64. The portion 60 defines a passageway or internal passage portion 66 extending between the first end 62 and the second end 64 of the portion 60.

As illustrated in FIG. 1, portions of the suture strand 50 extend through the internal passage portion 66. The suture strand 50 passes through the internal passage portion 66 a first time to define the first adjustable loop 56, and a second time to define the second adjustable loop 58. The internal passage portion 66 can be any suitable passage through the suture strand 50, through which portions of the suture strand 50 can pass to define the first and the second adjustable loops 56 and 58, such as the passage portions described in the '927 Application and the '647 Patent, which are incorporated herein by reference.

Slidably mounted to the first adjustable loop 56 is a first suture tail 70a. Slidably mounted to the second adjustable loop 58 is a second suture tail 70b. The first suture tail 70a is similar to the second suture tail 70b. In order to facilitate differentiation between the first suture tail 70a and the second suture tail 70b, such as by a surgeon in the operating room, the first suture tail 70a and the second suture tail 70b can include any suitable distinguishing identifiers. For example, the first and second suture tails 70a and 70b can be provided with different colors, different visual patterns, different indicia (such as different striping) different sizes (such as different lengths or widths), or different materials.

The first suture tail 70a and the second suture tail 70b can each be made of any suitable material, such as the same material as the suture strand 50, or any of the alterative materials of the suture strand 50 described above. Features in common between the first and second suture tails 70a and 70b are illustrated and described using similar reference numbers, but reference numbers of the first suture tail 70a include the suffix “a,” and reference numbers of the second suture tail 70b include the suffix “b.” Because the first suture tail 70a is similar to the second suture tail 70b, the detailed description below of the first suture tail 70a describes the second suture tail 70b as well.

The first suture tail 70a includes a first end 72a and a second end 74a. The first end 72a is opposite to the second end 74a. At the first end 72a is a locking portion 76a. Extending from the locking portion 76a to the second end 74a is an elongated tail portion 78a. The locking portion 76a includes a portion 80a, which defines a passageway or passage portion 86a. The passage portion 86a extends between a first opening 82a and a second opening 84a formed between suture braids of the first suture tail 70a. The first adjustable loop 56 extends through the passage portion 86a, thereby coupling the first suture tail 70a to the suture strand 50. Similarly, the second suture tail 70b can be arranged such that the suture strand 50 of the second adjustable loop 58 extends through passageway 86b defined by the second suture tail 70b at the locking portion 76b.

As further described herein, the construct 14 is configured such that upon pulling the first end 52 of the suture strand 50, a portion of the suture strand 50 within the internal passage portion 66 will be pulled out through the first sleeve end 62 thereof, thus closing the first adjustable loop 56, and reducing the size thereof. Similarly, pulling the second end 54 pulls a portion of the suture strand 50 out of the internal passage portion 66 at the second sleeve end 64, in order to reduce the size of, and close, the second adjustable loop 58. As the first and second ends 52 and 54 are pulled, the first and second adjustable loops 56 and 58 are tensioned, thus causing the internal passage portion 66 to collapse onto portions of the suture strand 50 extending therethrough, thereby restricting movement of the suture strand 50 through the sleeve internal passage 66, and thus locking the first and second adjustable loops 56 and 58 to prevent them from reopening. In this manner, the first and the second adjustable loops 56 and 58 are self-locking.

Prior to the tissue fixation device 10 being implanted, the first and second adjustable loops 56 and 58 can be provided in a retracted or closed position, such that the first and second adjustable loops 56 and 58 are small, which can advantageously reduce any possibility of tangling of the first and second adjustable loops 56 and 58. In order to open the first and second adjustable loops 56 and 58, the first suture tail 70a can be pulled at the second end 74a, thereby pulling a portion of the suture strand 50 out from within the internal passage portion 66 at the second sleeve end 64. The second adjustable loop 58 can be opened by pulling the second suture tail 70b, such as at the second end 74b, in order to pull a portion of the suture strand 50 out from within the internal passage portion 66 at the first sleeve end 62. Because the first and the second adjustable loops 56 and 58 have not yet been tensioned, such as when closed against tissue 120 to secure the tissue 120 to bone 110 as described herein, and the anchor 12 has not yet collapsed as illustrated in FIG. 7 for example, the first and the second adjustable loops 56 and 58 can be opened in this manner.

With reference to FIG. 2, the tissue fixation device 10 is illustrated implanted in bone 110. The bone 110 includes a relatively soft inner cancellous bone layer 112, and a relatively hard outer cortical bone layer 114. An outer surface 116 of the bone 110 is at a side of the cortical bone layer 114 opposite to the cancellous bone layer 112. Formed within the bone 110 is a bone hole 118. The bone hole 118 is formed through the outer surface 116, through the outer cortical bone layer 114, and into the cancellous bone layer 112. The bone hole 118 can be formed in any suitable manner, such as with a suitable drill or impactor, or any other suitable bone cutting device.

The tissue fixation device 10 is arranged such that the anchor 12 is seated in the bone hole 118. The anchor 12 protrudes slightly into the cancellous bone layer 112 proximate to the cortical bone layer 114 at the first and second anchor ends 20 and 22 thereof. The first and the second end surfaces 30 and 32 abut the cortical bone layer 114 at a side thereof opposite to the outer surface 116 and adjacent to the cancellous bone layer 112. As illustrated in FIG. 2, the first and second adjustable loops 56 and 58 are made smaller or closed, so as to reduce any possibility of tangling, as described above. After one or more of the tissue fixation devices 10 are implanted in bone, they may be used to secure tissue 120 to bone 110, as will now be described.

With reference to FIG. 3, two of the tissue fixation devices 10 are illustrated implanted in the bone 110 in the same manner as illustrated in FIG. 2. The tissue fixation devices 10 can be used to secure tissue 120 to the bone 110. The two tissue fixation devices 10 are illustrated as first tissue fixation device 10a and second tissue fixation device 10b. Only the first tissue fixation device 10a need be used to secure tissue 120 to bone 110. To enhance fixation of the tissue 120 to bone 110, however, additional tissue fixation devices 10 can be used, such as the second tissue fixation device 10b or yet additional tissue fixation devices 10, as described herein.

The second tissue fixation device 10b is substantially similar to, or the same as, the first tissue fixation device 10a. The second tissue fixation device 10b includes first and second suture tails 70c and 70d, which are similar to, or the same as, the first and second suture tails 70a and 70b of the first tissue fixation device 10a. The first and second adjustable loops of the second tissue fixation device 10b are illustrated at reference numerals 56b and 58b respectively.

After the tissue fixation devices 10a and 10b are initially seated in the bone hole 118 as illustrated in FIG. 2, their suture tails 70a, 70b, 70c, and 70d are pulled through the tissue 120, as illustrated in FIG. 3. The tails 70a, 70b, 70c, and 70d and the loops 56a, 58a, 56b, and 58b are then arranged as described below and illustrated in FIGS. 4-7 to apply pressure against the tissue 120 and secure the tissue 120 against the outer bone surface 116.

FIGS. 4-7 illustrate arrangement of the first and second tails 70a and 70b, and the first and second loops 56a and 56b, of tissue fixation device 10 (illustrated in FIG. 3 at reference number 10a to distinguish the second tissue fixation device 10b) to retain the tissue 120 against the outer bone surface 116. The method of FIGS. 4-7 applies to both the first and second tissue fixation devices 10a and 10b of FIG. 3.

After the tissue fixation device 10 has been seated in the bone hole 118 as illustrated in FIG. 2, the first and second suture tails 70a and 70b are pulled through the tissue 120, thus pulling the first and second adjustable loops 56 and 58 through the tissue 120 as well, as initially set forth above. The first and second suture tails 70a and 70b, and the first and second adjustable loops 56 and 58 associated therewith, are pulled through spaced apart areas of the tissue 120, thus leaving a portion of the tissue 120 between the first and second adjustable loops 56 and 58.

With reference to FIG. 4, after the first and second adjustable loops 56 and 58 are pulled through the tissue 120 using the first and second suture tails 70a and 70b, the first and second adjustable loops 56 and 58 can be coupled together and cinched down against the tissue 120 in order to secure the tissue against the outer surface 116 of the bone 110. For example, the second end 74a of the first suture tail 70a can be pulled through the second adjustable loop 58 in order to pull the first suture tail 70a through the second adjustable loop 58, and pull the first adjustable loop 56 such that a portion thereof extends through the second adjustable loop 58, as illustrated in FIG. 5.

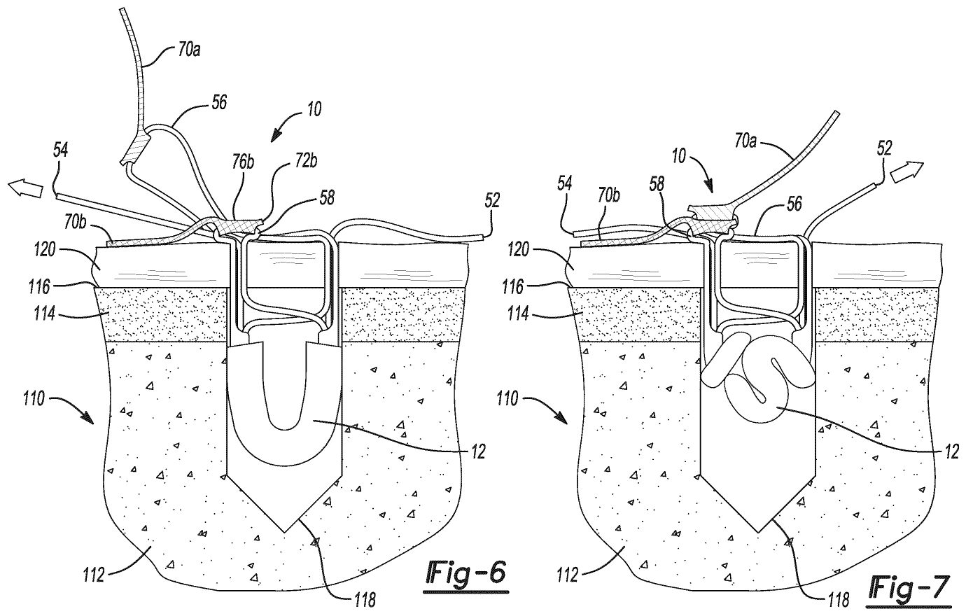

With reference to FIG. 6, the second adjustable loop 58 is cinched down onto the first adjustable loop 56 by pulling on the second end 54. As the second end 54 is pulled, the second adjustable loop 58 will reduce in size and pull the first adjustable loop 56 against the tissue 120, and affix the tissue 120 to the outer surface 116 of the bone 110. With reference to FIG. 7, after the size of the second adjustable loop 58 is reduced, the size of the first adjustable loop 56 is reduced by pulling the first end 52 of the suture strand 50. As the first adjustable loop 56 is reduced in size, the first suture tail 70a will abut the second suture tail 70b and/or the second adjustable loop 58. As the first end 52 is pulled further, the first suture tail 70a and/or the first adjustable loop 56 will pull the second suture tail 70b and the second adjustable loop 58 further against the tissue 120, in order to further secure the tissue 120 to the outer surface 116 of the bone 110. As the first and second ends 52 and 54 are pulled as illustrated in FIGS. 6 and 7 respectively, the first and second adjustable loops 56 and 58 are tensioned, thus causing the internal passage portion 66 to collapse onto portions of the suture strand 50 extending therethrough, thereby restricting movement of the suture strand 50 through the internal passage portion 66, and thus locking the first and second adjustable loops 56 and 58 to prevent them from reopening. The anchor 12 will then deform as illustrated in FIG. 7 to lock the anchor 12 in the bone hole 118.

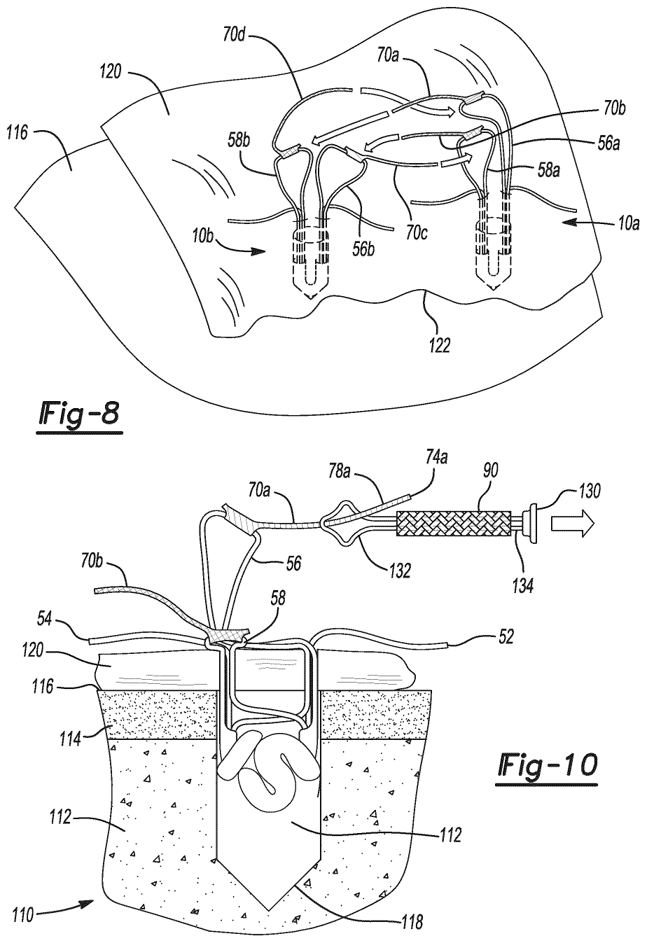

Although FIGS. 3-7 illustrate coupling together first and second adjustable loops 56 and 58 from the same tissue fixation device 10, various other configurations and arrangements are contemplated. For example, and with reference to FIG. 8, in applications where both the first tissue fixation device 10a and the second tissue fixation device 10b are used, the first suture tail 70a of the first tissue fixation device 10a can be coupled to the second tissue fixation device 10b. This can be done by inserting the first suture tail 70a through the second adjustable loop 58b of the second tissue fixation device 10b, which will couple the first adjustable loop 56a of the first tissue fixation device 10a to the second adjustable loop 58b of the second tissue fixation device 10b once the second adjustable loop 58b is cinched down onto the first adjustable loop 56a in the same manner described above. Similarly, the second adjustable loop 58b of the second tissue fixation device 10b can be coupled to the first adjustable loop 56a of the first tissue fixation device 10a by closing the second adjustable loop 58a onto the first adjustable loop 56b as described above. Use of both the first and second tissue fixation devices 10a and 10b to fix the tissue 120 to the bone 110 advantageously retains a greater portion of the tissue 120 against the bone 110, thereby distributing load against the tissue 120 and decreasing any possibility of the suture strands 50 cutting through the tissue 120.

With reference to FIG. 9, a third tissue fixation device 10c can be used to affix the tissue 120 to the bone 110. The third tissue fixation device 10c can be similar to, or the same as, the first and second tissue fixation devices 10a and 10b. The third tissue fixation device 10c can be implanted in the bone 110 spaced apart from the tissue 120, and beyond a distal end 122 thereof.

The third tissue fixation device 10c can be coupled with the first and second tissue fixations device 10a and 10b in any suitable manner. For example, the first suture tail 70a of the first tissue fixation device 10a can be inserted through a first adjustable loop 56c of the third tissue fixation device 10c in order to couple the first adjustable loops 56a and 56c together after the first adjustable loop 56c is cinched down onto the first adjustable loop 56a of the first tissue fixation device 10a. The second adjustable loop 58c of the third tissue fixation device 10c can be coupled to the second adjustable loop 58b of the second tissue fixation device 10b, such as by inserting the second suture tail 70d through the second adjustable loop 58c and closing the second adjustable loop 58c onto the second adjustable loop 58b. The first and second tissue fixation devices 10a and 10b can be connected by coupling the second adjustable loop 58a of the first tissue fixation device 10a with the first adjustable loop 56b of the second tissue fixation device 10b as described above. Thus, the suture tails 70a-70f are coupled to loops 56a-56c and 58a-58c of neighboring tissue fixation devices 10a-10c in order to chain the tissue fixation devices 10a-10c together. Including the third tissue fixation device 10c in the arrangement of FIG. 9 advantageously secures the distal end 122 of the tissue 120 to the outer surface 116 of the bone 110.

With reference to FIGS. 10 and 11, an additional soft or flexible anchor 90 may be added to the tissue fixation device 10 in order to provide additional fixation of the tissue 120. The anchor 90 may be similar to the anchor 12, or any other suitable soft or flexible anchor that is deformable in order to be retained in the bone hole 124. The anchor 90 can be added to the tissue fixation device 10 in any suitable manner, such as with a threader 130 illustrated in FIG. 10. The threader 130 includes a loop portion 132 at a distal end of elongated rods 134. The anchor 90 is initially seated on the elongated rods 134 such that the elongated rods 134 extend through the anchor 90.

The anchor 90 can be attached to the first suture tail 70a by first passing the tail portion 78a thereof into the loop portion 132, and folding the tail portion 78a over the loop portion 132. The anchor 90 is then slid over the tail portion 78a that is folded over the loop portion 132, and onto the first suture tail 70a. The first suture tail 70a and the first adjustable loop 56 coupled thereto are then pulled through the anchor 90 so that the anchor 90 is seated on the first adjustable loop 56. The anchor 90 can be implanted in the bone 110 in bone hole 124 beyond the distal end 122 of the tissue 120, as illustrated in FIG. 11. As the first end 52 of the suture strand 50 is pulled, the first adjustable loop 56 will shorten, thus drawing the first suture tail 70a against the anchor 90, and deforming the anchor 90 so that the anchor 90 will be retained in the bone 110. The first adjustable loop 56 will also span and contact the distal end 122 of the tissue 120 in order to retain the distal end 122 to the bore 110. The first end 72a of the first suture tail 70a will catch on an end of the anchor 90 and the first adjustable loop 56 will pull the second adjustable loop 58, as well as the tissue 120, towards the anchor 90, thereby tensioning the tissue 120 in the anterior to posterior direction, for example.

With reference to FIG. 12, another tissue fixation device according to the present teachings is illustrated at numeral 210. The tissue fixation device 210 generally includes a soft or flexible anchor 212 and a self-locking construct 214. The soft or flexible anchor 212 can be any suitable anchor, such as the anchor 12 described above, or any suitable alternate configurations thereof, such as the alternate configurations described above. The anchor 212 generally includes a first end 220 and a second end 222, which is opposite to the first end 220. The anchor 212 defines a first opening 224 at the first end 220, and a second opening 226 at the second end 222. An anchor internal passage 228 extends through the anchor 212 between the first opening 224 and the second opening 226.

The self-locking construct 214 can be any suitable self-locking construct, such as a construct including a suture 250. Alternately, the construct 214 can be any of the alternate constructs described above with respect to the construct 14. The suture 250 generally includes a first end 252 and a second end 254, which is opposite to the first end 252. The construct 214 defines a first loop 256 and a second loop 258, which are formed as described below.

A first body portion 260 of the suture 250 is generally between the first loop 256 and the second loop 258, and defines a passageway 262 extending between a first end 264 and a second end 266, through which the suture 250 extends to define the first loop 256. The first loop 256 extends from the first body portion 260. The suture 250 further includes a second body portion 270 proximate to the second end 254. The second body portion 270 defines a passageway 272 extending between a first opening 274 and a second opening 276 through which the suture 250 extends to define the second loop 258. The second loop 258 extends from the second body portion 270. The anchor 212 is seated on the first loop 256, such that portions of the suture 250 defining the first loop 256 extend through the anchor internal passage 228.

The tissue fixation device 210 further includes a first tail 280a and a second tail 280b. The first tail 280a is coupled to the first loop 256, and the second tail 280b is coupled to the second loop 258. The first and second tails 280a and 280b can be made of any suitable material, such as braided hollow-core suture. The first and second tails 280a and 280b can also be made of the same material that the first and second suture tails 70a and 70b of FIG. 1 are made of.

The first tail 280a is substantially similar to, or the same as, the second tail 280b. Therefore, the description of the first tail 280a also applies to the second tail 280b. Features in common between the first tail 280a and the second tail 280b are illustrated and described using like reference numbers, with the features of the first tail 280a including the suffix “a” and the features of the second tail 280b including the suffix “b.” To facilitate distinguishing between the first and second tails 280a and 280b, such as by a surgeon in an operating room, the first and second tails 280a and 280b can include any suitable distinguishing features. For example, the first tail 280a can have a different color, pattern, design, length or width as compared to the second tail 280b.

The first tail 280a includes a first end 282a and a second end 284a, which is opposite to the first end 282a. Proximate to the first end 282a is a locking member/portion 286a. Extending from the locking member/portion 286a to the second end 284a is a first tail portion 288a. The first tail 280a defines a passageway 290a at the locking member portion 286a. The passageway 290a extends between a first opening 292a and a second opening 294a of the first tail 280a. The first loop 256 extends through the passageway 290a. The first tail 280a and the second tail 280b can be used to adjust the sizes of the first and the second loops 256 and 258 respectively, as described herein with respect to FIGS. 22A-22C.

The first loop 256 can be made smaller by pulling the first end 252 through the passageway 262, thereby pulling a portion of the suture 250 proximate to the first end 252 through the passageway 262. The second loop 258 can be made smaller by pulling the second end 284b of the second tail 280b away from the passageway 272 so as to slide the second body portion 270 towards the second tail 280b. Although the second loop 258 is illustrated as an adjustable loop, the second loop 258 can be a rigid loop fixedly secure at the second body portion 270, for example, so as to restrict the second loop 258 from opening or closing.

The tissue fixation device 210 can be implanted in bone 110 in any suitable manner, such as described above with respect to the tissue fixation device 10 in FIG. 2. The tissue fixation device 210 can be used to secure tissue 120 to the bone 110 in any suitable manner, such as described above with respect to the tissue fixation device 10. For example, the anchor 212 can be seated into the bone hole 118 and then the first and second tails 280a and 280b can be passed through the tissue 120. The first tail 280a can be passed through the second loop 258, so as to arrange the first loop 256 such that a portion thereof extends through the second loop 258. The first loop 256 can be made smaller so as to press against and onto the tissue 120 by pulling the first end 252, which reduces the length of the first loop 256.

As the first end 252 is pulled, the locking member/portion 286a engages the second loop 258, so as to prevent the first tail 280a from passing back through the second loop 258. Furthermore, tension between the portion of the suture 250 extending through the passageway 262 and an interior of the passageway 262 causes the passageway 262 to collapse on the suture 250 and prevent the first loop 256 from reopening. If the second loop 258 is provided as a flexible loop, the second loop 258 may be made smaller by pulling the second tail 280b. Tension between the portion of the suture 250 extending through the passageway 272 and an interior of the passageway 272 cause the passageway 272 to collapse onto the suture 250 and prevent the second loop 258 from reopening.

Although FIG. 12 illustrates the tissue fixation device 210 with the anchor 212 on the first loop 256, the anchor 212 of the tissue fixation device 210 can be initially seated on the first tail 280a, as illustrated in FIG. 13, such that the first tail portion 288a extends through the anchor internal passage 228. With reference to FIGS. 14-18, use of the tissue fixation device 21 of FIG. 13 with the anchor 212 initially seated on the first tail 280a to secure tissue 120 to the bone 110 will now be described. In applications where the anchor 212 is initially seated on the first loop 256, as illustrated in FIG. 12, the method of FIGS. 14-18 may also be used to secure tissue 120 to bone 110.

The anchor 212 is initially implanted in the bone hole 118, and the first tail 280a is passed through the tissue 120. A cannula 310 is arranged such that it extends through a patient's outer skin layer to facilitate introduction of the tissue fixation device 210 to an implant site, and to facilitate manipulation of the tissue fixation device 210 outside of the patient's body. By arranging the anchor 212 on the first tail 280a as opposed to on the first loop 256 as illustrated in FIG. 12, the construct 214 can remain outside of the joint space during much of the procedure to facilitate management of the fixation device 210, and particularly management of the construct 214.

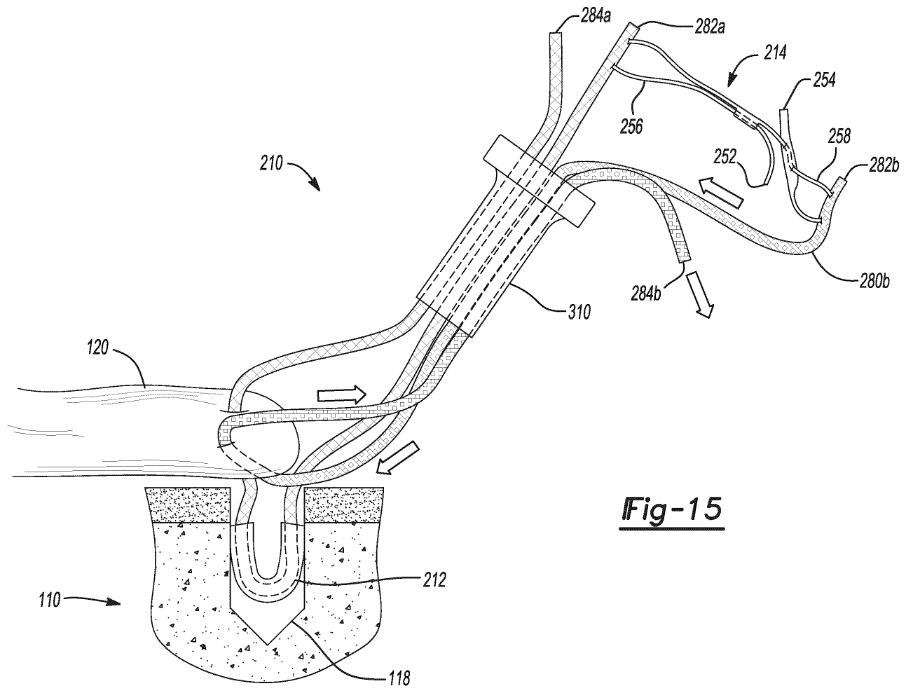

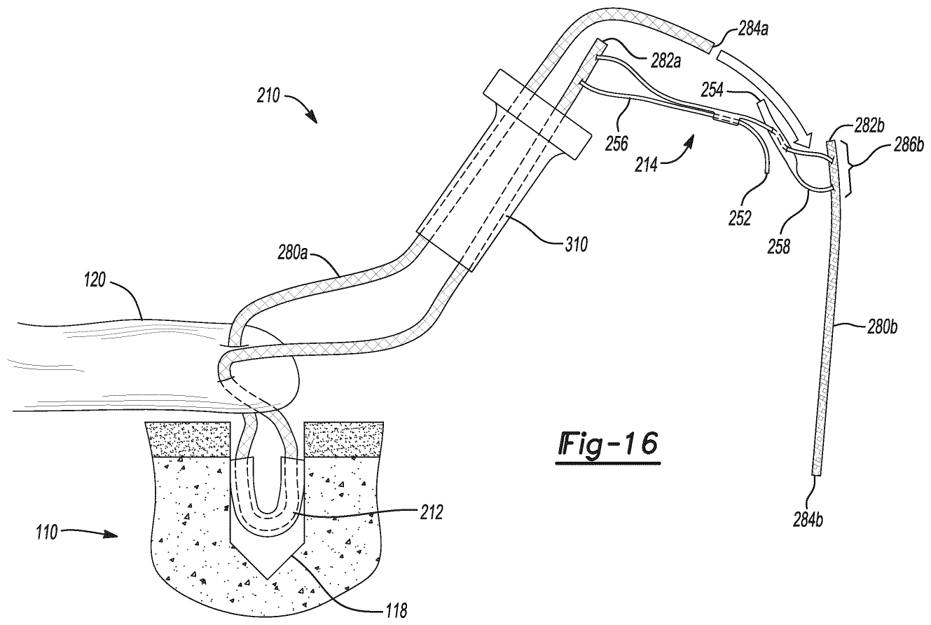

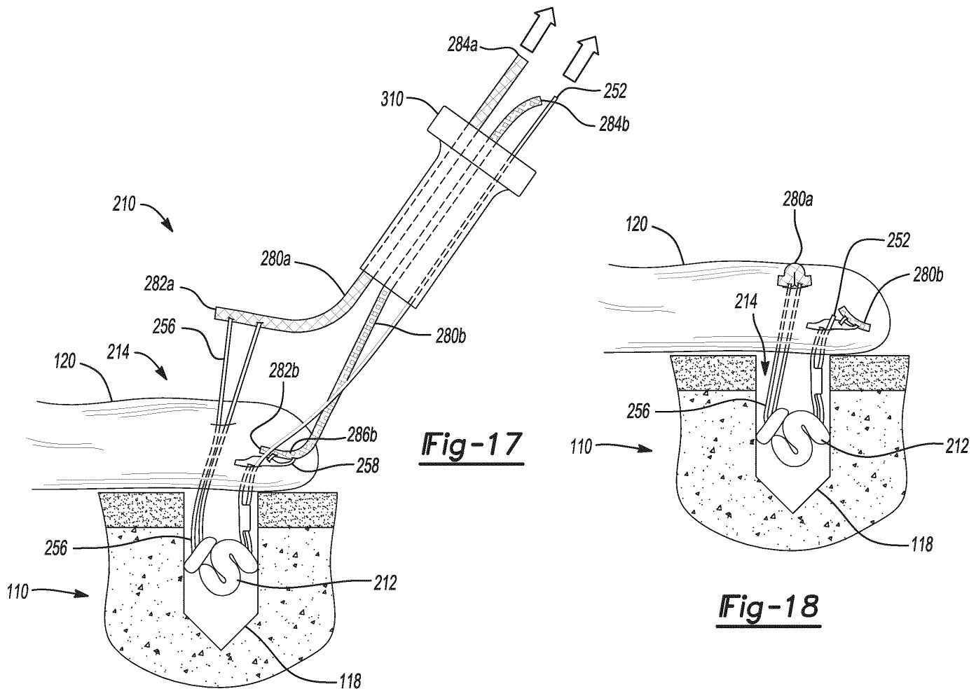

After the first tail 280a is passed through the tissue 120 as illustrated in FIG. 14, the second tail 280b is passed through the cannula 310, through the tissue 120, and back through the cannula 310, as illustrated in FIG. 15. With reference to FIG. 16, the first tail 280a is then passed through the second loop 258. Passing the first tail 280a through the second loop 258 draws the construct 214 through the cannula 310 and into engagement with the tissue 120, as illustrated in FIG. 17. More specifically, the first tail 280a is pulled until it completely passes through the tissue 120 and pulls the first loop 256 through the anchor 212 and up from the anchor 212 through the tissue 120. Pulling the first tail 280a also positions the second tail 280b at the tissue 120 such that the locking member/portion 286b abuts the tissue. To close the first loop 256 and secure the tissue 120 against the bone 110, the first end 252 of the suture 250 is pulled, as illustrated in FIG. 17. Excess portions of the first and second tails 280a and 280b can be removed after the first loop 256 has been completely zipped down, as illustrated in FIG. 18 for example. Thus, with the tissue fixation device 210, only a single suture strand need be pulled to close and tighten the construct 214 against the tissue 120.

A single tissue fixation device 210 can be used to secure the tissue 120 to the bone 110, or multiple tissue fixation devices 210 can be used. For example, multiple tissue fixation devices 210 can be individually implanted and not connected to each other, similar to the arrangement illustrated in FIG. 3 with respect to the tissue fixation device 10. Alternatively, multiple ones of the tissue fixation device 210 can be linked together, similar to the arrangement of FIGS. 3 and 8 with respect to the tissue fixation device 10. Thus, the first loop 256 of the tissue fixation device 210 can be coupled to the second loop 258 of a neighboring tissue fixation device 210 by inserting the first tail 280a through the second loop 258 of the neighboring tissue fixation device. Furthermore, the first or second tails 280a or 280b can be coupled to an anchor, which then may be implanted in bone, similar to that which is illustrated in FIG. 11 with respect to the tissue fixation device 10.

With additional reference to FIG. 19, the tissue fixation device 210 can include a second tail in the form of a braided strip 350. The braided strip 350 can be made of any suitable material, such as suture, or of any of the alternatives described above with respect to the suture strands 50 and 250. The braided strip 350 generally includes a flat braided portion 352 and a round braided portion 354, as viewed in cross-section, extending from the flat braided portion 352.

The flat braided portion 352 defines a first opening 356 and a second opening 358. As illustrated in FIG. 19, the second loop 258 passes through the first and second openings 356 and 358 in order to secure the braided strip to the second loop 258. However, the second loop 258 is optional and thus need not be included. When the tissue fixation device 210 does not include the second loop 258, the braided strip 350 can be coupled to the suture 250 without a loop, such as with a knot or any other suitable coupling configuration. The braided strip 350 may also be unitary with the rest of the suture 250.

Instead of passing the first tail 280a through the second loop 258, the first tail can be passed through the flat braided portion 352 of the braided strip 350. As a result, when the first loop 256 is made smaller, as was described above in connection with FIG. 17, the braided strip 350 will extend across the tissue 120 in order to dissipate load applied to the tissue 120 by the tissue fixation device 210. The round braided portion 354 expands the distance that the braided strip 350 will extend across the tissue 120, thereby further dissipating load.

FIG. 19 illustrates the braided strip 350 (without the round braided portion 354) as included with the tissue fixation device 210 prior to being introduced to an implant site. However, the braided strip 350 may be added to the fixation device 210 after the first loop 256 has been passed through the tissue, as illustrated at FIG. 20. For example, the braided strip 350 may be arranged at the second end 284a of the first tail 280a and at the second end 284b of the second tail 280b. The first and second ends 282a and 284b may be passed through the braided strip 350, and the braided strip 350 may be slid down and across each of the first and second tails 280a and 280b using a pusher device 380 until the braided strip 350 passes over the first ends 282a and 282b of the first and second tails 280a and 280b respectively. After the first end 252 of the suture 250 is pulled to close the first loop 256, excess portions of the first and second tails 280a and 280b may be removed, as illustrated in FIG. 21.

With reference to FIGS. 22A-22C, when the tissue fixation device 210 is provided with the second loop 258 as an adjustable loop, the second loop 258 can be moved from an intermediate position illustrated in FIG. 22A to a closed position illustrated in FIG. 22B by pulling a portion of the suture 250 extending beyond the second body portion 270 in the direction of the first end 252 (direction A), and pulling the second end 284b of the second tail 280b in generally the opposite direction (direction B). To reopen the second loop 258, the second end 254 of the suture 250 is pulled in direction C and the second end 284b of the second tail 280b is pulled in direction D, as illustrated in FIG. 22C. Being able to selectively open and close the second loop 258 is advantageous for a number of reasons. For example, if the second loop 258 is accidentally closed prematurely, the second loop 258 can easily be reopened, such as to permit passage of the first tail 280a therethrough.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

1-20. (canceled)

21. A method of securing soft tissue to bone, comprising:
delivering a distal end of an access cannula to a treatment site through a skin layer of a patient;
anchoring an adjustable suture construct at the treatment site, the adjustable suture construct including a first suture with a first free end that extends longitudinally through a first longitudinal passage in the first suture to form a first self-locking adjustable loop, the first free end pullable for reducing a size of the first self-locking adjustable loop, the adjustable suture construct further including a first flexible tail that is coupled to the first self-locking adjustable loop, wherein said anchoring leaves the pullable first free end of the first suture and a first tail end of the first flexible tail extending through the access cannula to a location outside the patient, the first tail end additionally passing through a soft tissue positioned over a bone at the treatment site;
threading, after said anchoring, the first tail end through a first piece of a flat material outside the patient; and
advancing the first piece of the flat material through the access cannula to the treatment site along the first flexible tail.

22. The method of claim 21, wherein said anchoring includes anchoring the adjustable suture construct with an anchoring member positioned at least partly inside a bone hole at the treatment site.

23. The method of claim 22, wherein the anchoring member comprises a soft anchor.

24. The method of claim 22, wherein the anchoring member comprises a flexible tubular anchor.

25. The method of claim 21, wherein said anchoring leaves at least part of the adjustable suture construct inside a bone hole at the treatment site.

26. The method of claim 21, wherein the adjustable suture construct additionally includes a second flexible tail coupled thereto, and wherein said anchoring additionally leaves a second tail end of the second flexible tail extending through the access cannula to the location outside the patient.

27. The method of claim 26, wherein said advancing additionally includes the first piece of the flat material being advanced through the access cannula along the second flexible tail

28. The method of claim 27, wherein the first piece of the flat material is advanced along the second flexible tail until the entirety of the second flexible tail has passed through the first piece of the flat material.

29. The method of claim 21, wherein said advancing includes pushing the first piece of the flat material through the access cannula with a pusher device.

30. The method of claim 21, wherein the first piece of the flat material comprises a strip of material.

31. The method of claim 21, wherein the first piece of the flat material comprises a braided material.

32. The method of claim 21 further comprising pulling on the first free end of the first suture as the first free end is extending through the access cannula to reduce a size of the first self-locking adjustable loop.

33. The method of claim 21 further comprising pulling on the first tail end of the first flexible tail as the first tail end is extending through the access cannula to increase a size of the first self-locking adjustable loop.

34. The method of claim 21 further comprising positioning the first piece of the flat material against the soft tissue at the treatment site.

35. The method of claim 34 further comprising pulling on the first free end of the first suture to tension the first piece of the flat material over the soft tissue.

36. The method of claim 35, wherein the first piece of the flat material is effective to dissipate a load applied to the soft tissue from said pulling.

37. The method of claim 21, wherein the first piece of the flat material is advanced along the first flexible tail until the entirety of the first flexible tail has passed through the first piece of the flat material.

38. The method of claim 21, wherein the first piece of the flat material is advanced along the first flexible tail until a portion of the first self-locking adjustable loop to which the first flexible tail is coupled passes through the first piece of the flat material.

39. The method of claim 21, wherein the first flexible tail comprises a hollow core suture.

40. The method of claim 39, wherein the first flexible tail being coupled to the first self-locking adjustable loop includes suture of the first self-locking adjustable loop extending longitudinally through an interior of the hollow core suture.
